(19) United States
(12) Patent Application Publication
(10) Pub. No.: US 2013/0268043 A1
(43) Pub. Date: Oct. 10, 2013

(54) Stimulating Device

(71) Applicants: Claudia Tasche, Elanora Heights (AU); John Parker, Roseville (AU); Dusan Milojevic, Westleigh (AU); Colin Irwin, Artarmon (AU)

(72) Inventors: Claudia Tasche, Elanora Heights (AU); John Parker, Roseville (AU); Dusan Milojevic, Westleigh (AU); Colin Irwin, Artarmon (AU)

(21) Appl. No.: 13/659,531

(22) Filed: Oct. 24, 2012

Related U.S. Application Data
(63) Continuation of application No. 11/045,624, filed on Jan. 28, 2005, now abandoned.
Application No. 11/045,624 is a continuation-in-part of application No. 10/494,995, filed on Sep. 23, 2004.

(30) Foreign Application Priority Data
Nov. 9, 2001 (AU) AU PR 8792

(51) Int. Cl. A61N 1/05

(52) U.S. Cl. CPC A61N 1/0551
USPC 607/137

(57) ABSTRACT

An implantable apparatus, such as a inner ear prosthetic hearing implant, and a method for delivering neuron firing threshold-reducing stimuli to a neural network of an implantee are provided. The apparatus comprises a stimulator device that generates stimulation signals, and an electrode array that receives the stimulation signals and delivers the stimuli to the neural network of the implantee in response to the signals. The stimuli delivered to the implantee facilitates and/or controls the production and/or release of naturally occurring agents into the neural network to reduce the firing thresholds of neurons.

### CROSS-REFERENCE TO RELATED APPLICATIONS

This application is a continuation of U.S. patent application Ser. No. 11/045,624, entitled “A Stimulating Device”, filed on Jan. 28, 2005, which is a continuation-in-part of and claims priority to U.S. patent application Ser. No. 10/494,995, entitled “Subthreshold Stimulation of a Cochlea,” filed May 7, 2004, which is a national stage application of PCT/AU02/01537, filed Nov. 11, 2002, which claims priority to Australian Provisional Application No. AU PR 8792, filed Nov. 9, 2001, the entire contents and disclosures of which are hereby incorporated by reference herein.

### BACKGROUND

The use of implantable medical devices to provide electrical stimulation therapy to individuals for various medical conditions has become more widespread in recent times. This has occurred as the advantages and benefits such devices provide become more widely appreciated and accepted throughout the population.

Electrical stimulation therapy can be used to deliver electrical stimulation to various locations within the body, and for a variety of purposes. For example, function electrical stimulation (FES) systems may be used to deliver electrical pulses to certain neurons of a recipient to cause a controlled movement of a limb of such a recipient.

A further type of medical device is an implantable hearing prosthesis system (IHPS). An IHPS can provide the benefit of hearing to individuals suffering from severe to profound sensorineural hearing loss. Sensorineural hearing loss is due to the absence or destruction of the hair cells in the cochlea which transduce acoustic signals into nerve impulses. An IHPS essentially simulates the cochlear hair cells by delivering electrical stimulation to the auditory nerve fibers. This causes the brain to perceive a hearing sensation resembling the natural hearing sensation.

It is generally desirable that electrical stimulation systems such as the noted IHPSs consume minimal power. Lower power consumption leads to smaller components and longer battery life.

In the case of an IHPS, attempts have been made to reduce the power consumption through the development of more efficient speech coding strategies. Other proposals have included positioning the stimulation electrodes closer to the neurons in the cochlea. These methods have been used with varying success.

It is desired to improve upon existing arrangements.

### SUMMARY

According to a first broad aspect of the present invention, there is provided an implantable apparatus for delivering electrical stimuli to an implantee. The apparatus comprises a stimulator that generates stimulation signals; and at least one electrode member for receiving the stimulation signals and for delivering the stimuli to the implantee in response to said signals; wherein the stimuli includes neuron firing threshold-reducing stimuli facilitating the production and/or release of naturally occurring agents to reduce the firing thresholds of neurons.

According to a second broad aspect of the present invention, there is provided a method of delivering stimuli to a neural network of an implantee, comprising: positioning at least one electrode member in a position suitable to deliver said stimuli to said implantee; generating stimulation signals; transmitting said signals to said at least one electrode member; and delivering said stimuli in response to said signals, wherein said stimuli includes neuron firing threshold-reducing stimuli having a magnitude below a perception threshold of the implantee, the neuron firing threshold-reducing stimuli facilitating the production and/or release of naturally occurring agents into the neural network to reduce the firing thresholds of neurons.

According to a third broad aspect of the present invention, there is provided a method of improving the efficacy of a prosthetic implant implanted in an implantee, comprising: generating stimulation signals; transmitting said signals to at least one electrode member positioned to deliver stimuli to the implantee in response to said signals; and delivering said stimuli in response to said signals, wherein said stimuli includes neuron firing threshold-reducing stimuli having a magnitude below a perception threshold of the implantee, the neuron firing threshold-reducing stimuli facilitating the production and/or release of naturally occurring agents into the neural network to reduce the firing thresholds of neurons and thus to reduce power consumption of said prosthetic implant.

### BRIEF DESCRIPTION OF THE DRAWINGS

FIG. 1 is a pictorial view of an implantable hearing prosthesis system (IHPS) and a clinician's computer suitable for implementing embodiments of the present invention.

FIG. 2 is a plan view of an implantable housing for an IHPS suitable for implementing embodiments of the present invention.

FIG. 2a is a cross-sectional view of the housing of FIG. 2 through line A-A of the housing illustrated in FIG. 2.

FIG. 2b is a further cross-sectional view of the housing of FIG. 2 through line B-B of the housing illustrated in FIG. 2.

FIG. 3 is an exemplary depiction of patterned electrical stimuli as a function of time.

FIG. 4 is another exemplary depiction of patterned electrical stimuli as a function of time.

FIG. 5 is another exemplary depiction of patterned electrical stimuli across multiple channels as a function of time.

FIG. 6 is another exemplary depiction of patterned electrical stimuli across multiple channels as a function of time.

FIG. 7 is another exemplary depiction of patterned electrical stimuli across multiple channels as a function of time.

FIG. 8 is another exemplary depiction of patterned electrical stimuli across multiple channels as a function of time.

FIG. 9 is a simplified drawing of another example of an implant according to one embodiment of the present invention.

FIG. 10 is a simplified drawing of another implant according to one embodiment of the present invention.

FIG. 11 is a functional block diagram of an exemplary stimulation system in accordance with one embodiment of the present invention.

FIG. 11A is a functional block diagram of a portion of the stimulation system illustrated in FIG. 11.

FIG. 12 shows a typical eABR recorded from a deaf guinea pig cochlea.

FIG. 13 is a schematic diagram of a guinea pig electrode array for delivering pharmacological agents to the scala tympani via two independent external pumps connected to a micro-tube assembly.

FIGS. 14a and 14b show eABR responses before and after perfusion with a BDNF solution according to one embodiment of the present invention.

FIG. 15 a graph showing results obtained by embodiments of the present invention relating to absolute eABR thresholds.

FIG. 16 is a graph showing results obtained by embodiments of the present invention relating to normalized eABR thresholds.

FIG. 17 is a graph showing comparative results of normalized eABR values before and after perfusion of RAP.

FIG. 18 is a graph showing comparative results of absolute eABR thresholds before and after perfusion with BDNF.

FIG. 19 is a graph showing comparative results of absolute eABR thresholds before and after perfusion with BDNF.

FIG. 20 is a graph showing comparative results of normalized eABR thresholds before and after perfusion with BDNF.

FIG. 21 is a table showing comparative experimental results achieved by one embodiment of the present invention.

FIG. 22 is a graph showing a comparison of psychophysical measures of threshold levels with behavioral measures of threshold levels in accordance with one embodiment of the present invention.

### DETAILED DESCRIPTION

Before describing embodiments of the present invention in detail, it is convenient to briefly review the general operation of an intra-cochlea implantable hearing prosthesis system (IHPS).

An IHPS bypasses the hair cells in the cochlea and delivers electrical stimulation to the auditory nerve fibers, thereby allowing the brain to perceive a hearing sensation resembling a natural hearing sensation. A variety of IHPSs are described in U.S. Pat. Nos. 4,532,930, 6,537,200, 6,565,503, 6,575,894 and 6,697,674, the entire contents and disclosures of which are hereby incorporated by reference herein.

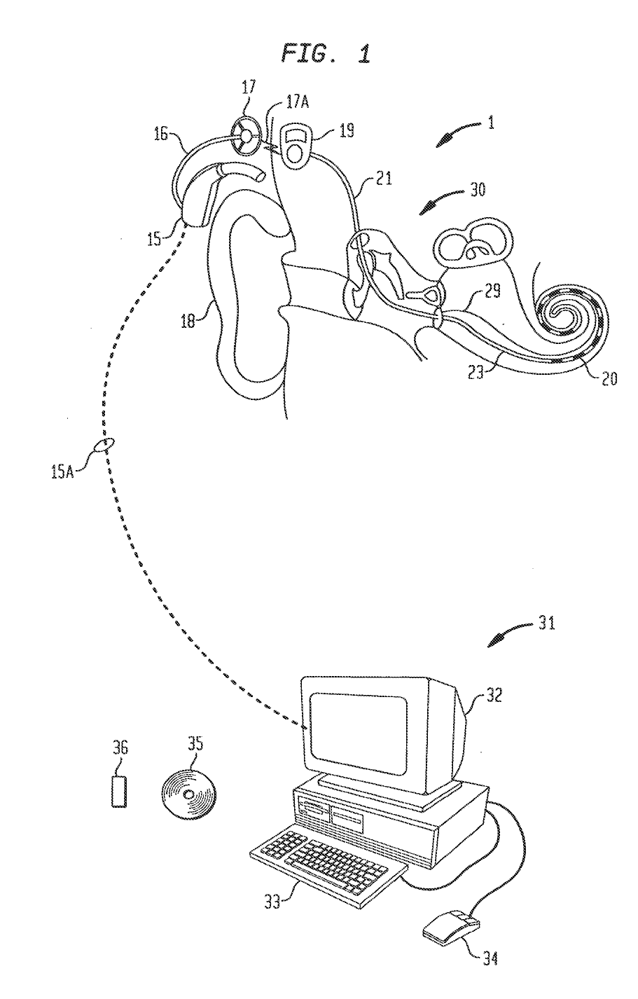

FIG. 1 is a pictorial view of an IHPS and a clinician's computer suitable for implementing embodiments of the present invention. In the arrangement illustrated in FIG. 1, an IHPS 1 typically comprises an external speech processor unit 15 connected via a lead 16 to an antenna transmitter coil 17. The external speech processor unit 15 includes a microphone, electronics for performing speech processing, and a power source such as a rechargeable or non-rechargeable battery.

In this example, the speech processor unit 15 is configured to fit behind the outer ear 18. Alternatively, the speech processor unit 15 can be worn on the body such as in a pocket, a belt pouch or in a harness. Similarly, the microphone may be provided separately from the speech processor unit 15 and instead mounted on a clothing lapel, for example.

The IHPS 1 further includes an implantable receiver/stimulator unit (RSU) 19 connected to an electrode array 23 via a lead 21. The lead 21 includes individual wires extending from each electrode of the array 23 to the receiver/stimulator unit 19 to thus form separate channels.

The RSU 19 is implanted within a recess of the temporal bone and includes a receiver antenna coil for receiving power and data from the transmitter coil 17.

In operation, the electronics within the speech processor unit 15 converts sound detected by the microphone into a coded signal. The external antenna coil 17 transmits the coded signals, together with power, to the receiver/stimulator unit 19 via a radio frequency (RF) link 17A.

The antenna receiver coil receives the coded signal and power for the RSU 19 to process and output a stimulation signal to the electrode array 23.

Once implanted, implant assembly 30 of the IHPS is typically fitted/adjusted to suit the specific needs of the recipient. As the dynamic range for electrical stimulation is relatively narrow and varies across recipients and electrodes, there is a need to individually tailor the characteristics of electrical stimulation for each recipient. Behavioral measurements can be used to establish the useful range for each electrode, and such parameters can be stored within the recipient's speech processor unit 15 for continual use. This procedure is often referred to as “mapping” and is the term commonly given to the process of measuring and controlling the amount of electrical current delivered to the cochlea.

The mapping procedure is usually performed on a clinician's computer 31 shortly after surgical implantation of the implant assembly 30. The clinician's computer 31 is a general stand-alone personal computer including a screen 32, keyboard 33 and mouse 34. The computer 31 is loaded with a software program copied from, for example, a medium such as a compact disc (CD) 35 or a memory stick 36 into memory. The software program contains instructions that are carried out by a processor on the clinician's computer 31, to enable the clinician to perform the tests using a suitable interface when connected to the speech processor 15 via communication link 15A.

Exemplary embodiments of the present invention will now be described. With reference to FIG. 20, the present applicant has discovered and demonstrated that acute exogenous administration of Brain Derived Neurotropic Factor (BDNF) can lower firing thresholds of neurons in guinea pigs. This lowering of the firing thresholds was measured using neuro-physiological techniques. However, it will be appreciated that the lowering of firing thresholds may be similarly measured using behavioral techniques. Further details of these experiments are described below.

This finding of a method for reducing firing thresholds of spiral ganglion cells has in turn led to the development of an improved, more efficient, electrical stimulation system that consumes less power, due to a lowering of the firing thresholds of the neurons being stimulated.

The electrical stimulation device according to this disclosure facilitates the lowering of the firing thresholds of the neurons being stimulated, by creating conditions analogous to those used in the above-noted experiments, as will later be described in detail with reference to Example 1. In particular, the experimental conditions are replicated through exogenous and/or endogenous means in the electrical stimulation system. The adjustment of the BDNF levels required to achieve the lowering of thresholds, is enabled in one arrangement, through a feedback system described herein.

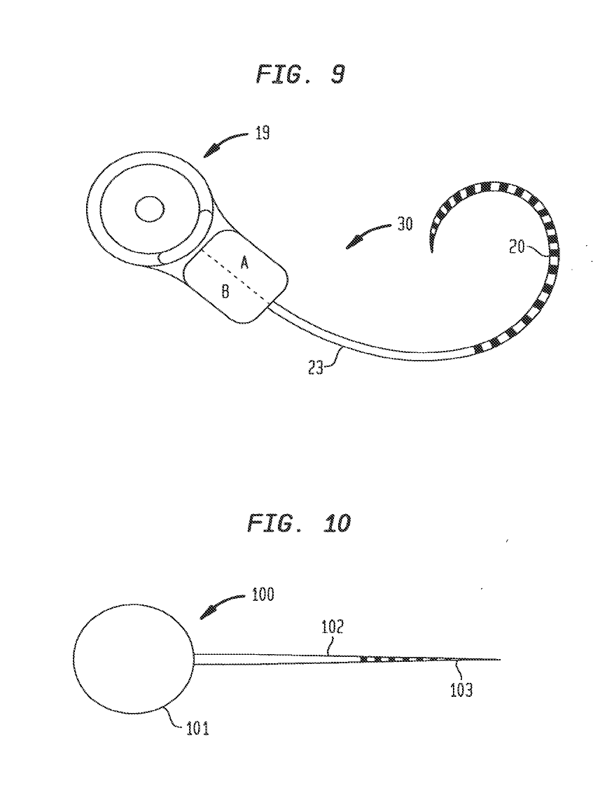

FIG. 9 is a simplified drawing of an exemplary implant according to one embodiment of the present invention. The implant assembly 30 illustrated in FIG. 9 comprises an RSU 19, as described above with reference to FIG. 1. A housing of the RSU 19 includes portion A and portion B. Portion A contains circuitry to enable the IHPS to deliver auditory informative stimuli according to conventional methods. Portion B contains circuitry to enable the IHPS to deliver patterned threshold-reducing electrical stimulation in accordance with the teachings of the present invention.

The relationship between patterned electrical stimulation and the release of endogenous Brain-Derived Neuroptrophic Factor (EBDNF) by sensory neurons is discussed in Activity-Dependent Release of Endogenous Brain-Derived Neurotrophic Factor from Primary Sensory Neurons Detected by ELISA In Situ, Balkowiec A. and Katz D. M., which is hereby incorporated by reference herein.

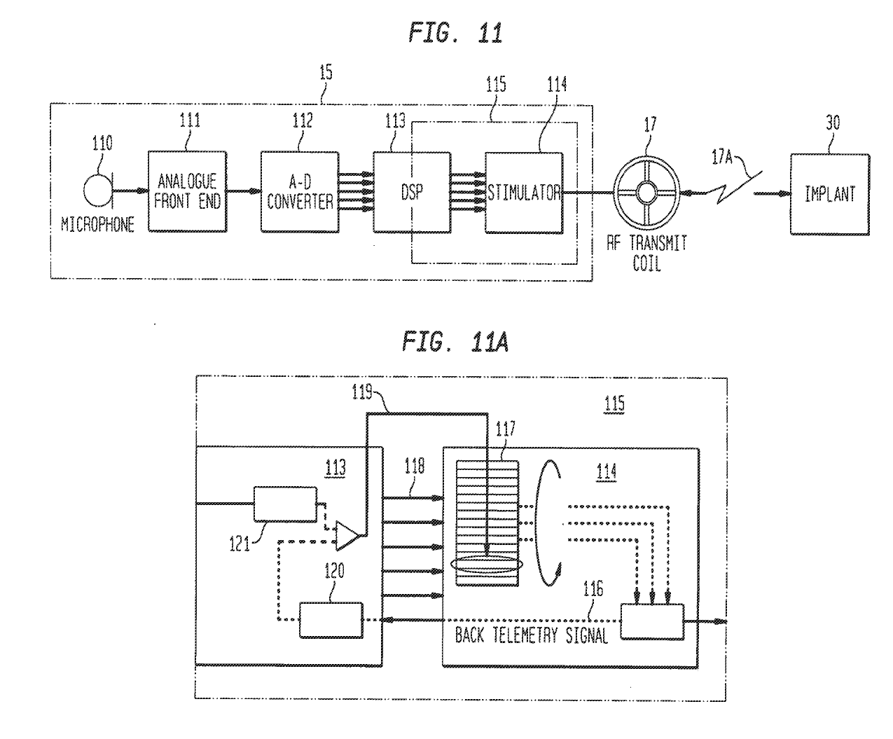

FIG. 11 is a functional block diagram of an exemplary stimulation system in accordance with one embodiment of the present invention. Referring to FIG. 11, the main functional blocks of the IHPS 1 include a microphone 110, an analog front end 111, an analog-to-digital converter (ADC) 112, a digital signal processor (DSP) 113, a stimulator 114 connected to the transmitter coil 17. The transmitter coil 17 communicates with the implant assembly 30 via the RF link 17A, as introduced above.

In operation, the DSP block 113 receives a signal from the microphone 110 and converts this signal into a data signal representing the auditory informative stimulation that is to be delivered by the implant 30. The DSP block 113 outputs the data signal which is then input in to the Stimulator block 114. The Stimulator block 114 converts the data signal into an RF signal which is then transmitted to, and decoded by, the implant 30 via the transmitter coil 17.

FIG. 11A is a functional block diagram of a portion 115 of the stimulation system illustrated in FIG. 11. In this example, the stimulator block 114 operates by continuously processing a script of commands 117. Typical commands include a command to retrieve the signal output 118 from the DSP block 113, and a command to send the necessary stimulus data to the implant 30. An exemplary script is presented in Listing 1. The script in Listing 1 is for a sound processing strategy where auditory informative stimuli are delivered on eight (8) of 22 electrodes in the electrode array, for each block of microphone input samples, which is known as an “8-maxima map.” The timing information detailed for each stimulus describes the time from the start of one stimulus to the start of the next stimulus, or the stimulus period. As the stimuli described here are charge balanced biphasic stimuli, the phase width and phase gap, if present, for each stimulus is selected as appropriate for the processing strategy.

Listing 1 - Typical stimulator block script

loop (forever)

retrieve DSP samples
deliver stimulus (DSP sample 1), 100 us
deliver stimulus (DSP sample 2), 100 us
deliver stimulus (DSP sample 3), 100 us
deliver stimulus (DSP sample 4), 100 us
deliver stimulus (DSP sample 5), 100 us
deliver stimulus (DSP sample 6), 100 us
deliver stimulus (DSP sample 7), 100 us
deliver stimulus (DSP sample 8), 100 us

endloop

In addition, In some embodiments, the IHPS 1 delivers patterned electrical stimulation, for the purpose of reducing the firing threshold of the neurons being stimulated. It is envisaged that this threshold-reducing patterned stimulation can delivered either on its own, or coincidentally with the processed audio signal stimulation. Both types of stimulation can be achieved by the IHPS 1 through a modification of the script used by the Stimulator block 114, with the amplitude of the threshold-reducing stimulation being preferably lower than the behavioral perception threshold of the implantee. The delivery of the threshold reducing stimulation alone can be advantageously delivered prior to the first “switch-on” of the recipient, and/or when the recipient is not listening to the processed audio signal, i.e., typically when the recipient is asleep, with an example of such a script provided in Listing 2.

Listing 2 - Typical stimulator block script

loop (forever)

deliver stimulus (sub-threshold), 50 ms

endloop

Meanwhile for the case where the threshold reducing patterned stimulation is delivered coincidentally with the processed audio signal stimulation, an example of a modified script is provided in Listing 3. Here the same processed audio signal stimulation is delivered as well as interleaved, threshold-reducing patterned electrical stimulation. Again, the timing information shown represents the period of each stimulus. The threshold-reducing patterned electrical stimulation typically uses a different phase width and phase gap, if present, compared to the auditory informative stimuli with the system described having the capability to deliver stimulation with time overlapping phases to different electrodes.

Listing 3 - Typical stimulator block script

loop (forever)

loop (3)

retrieve DSP samples
deliver stimulus (null), 50 us
deliver stimulus (DSP sample 1), 100 us
deliver stimulus (DSP sample 2), 100 us
deliver stimulus (DSP sample 3), 100 us
deliver stimulus (DSP sample 4), 100 us
deliver stimulus (DSP sample 5), 100 us
deliver stimulus (DSP sample 6), 100 us
deliver stimulus (DSP sample 7), 100 us
deliver stimulus (DSP sample 8), 50 us

endloop
deliver stimulus (sub-threshold), 50 ms

endloop

The release of BDNF from cultured cells can be correlated with certain parameters of patterned electrical stimulation. Balkoweic and Katz (incorporated by referenced above) applied patterned electrical field stimulation at 50 biphasic rectangular pulses of 25 msec, at 20 Hz, every 5 seconds to find increased extracellular BDNF levels by 20-fold, in comparison with cultures exposed for 30 minutes to continuous depolarization with elevated KCl. Moreover, Balkoweic and Katz found that the magnitude of BDNF release was dependent on the stimulus pattern and in particular, that high-frequency bursts are the most effective, thus showing that the optimal stimulus profile for BDNF release resembles that of other neuroactive peptides.

Hence, a starting point for the threshold-reducing patterned electrical stimulation for the IHPS 1, consists of 2 second, 50 Hz stimuli bursts delivered every 20 seconds. It will be understood that the exact parameters required for the threshold-reducing patterned electrical stimulation depends on individual circumstances, including the implemented speech coding strategy. Preferably, the amplitude of the threshold-reducing stimuli is less than a behavioral threshold value of the recipient or implantee.

The parameters or characteristics of the threshold-reducing patterned electrical stimulation can be varied, depending upon both how much reduction in the stimulation threshold is desired and for the individual implant recipient. The embodiments of the system described herein provide functionality to fine tune the characteristics of the threshold-reducing patterned electrical stimulation to suit these factors.

For the purpose of advantageously adjusting the characteristics of the threshold-reducing patterned electrical stimulation, a neurophysiological response feedback loop can be provided. However, in other arrangements, behavioral responses can be additionally or alternatively measured to monitor and adjust the characteristics of the threshold-reducing patterned electrical stimulation.

Referring again to FIG. 11A, a back telemetry path 116 is configured to receive a neural response measurement from the implant 30 via the RF link 17A. The neural response measurement is psychophysical in nature and is recorded from the auditory nerve, in response to applied electrical stimulation on any or more of the implant's electrodes. An example of this techniques is described in WO 2004/021885, assigned to the assignee of the present application, and incorporated by reference herein.

The measured back telemetry signal 116 is processed by a feedback processing block 120 and a resultant feedback/error signal is transmitted over path 119 to make adjustments to the script of commands 117.

Referring back to FIG. 11A, the momentary stimulation threshold levels of the neurons being stimulated can be measured, to thus determine the effectiveness of the patterned threshold-reducing stimuli being applied.

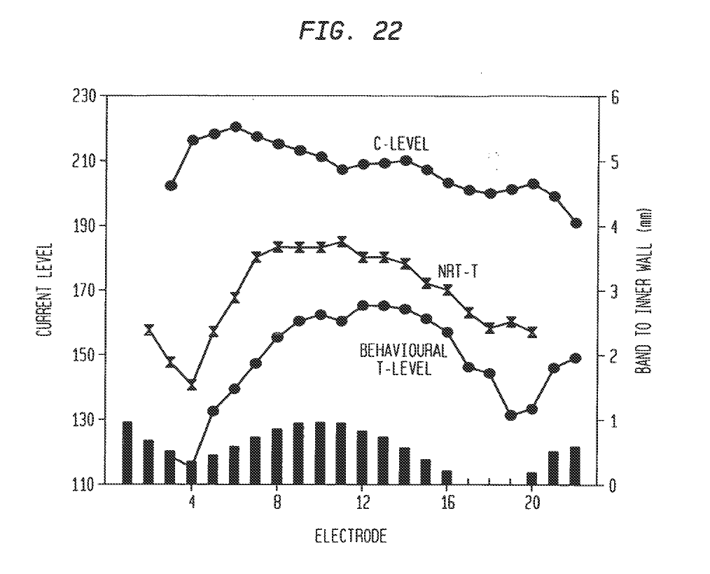

There is a correlation between a behavioral stimulation threshold level and the presence of a neural signal that is recorded in response to an applied momentary test signal. In conventional applications, this phenomena is used for mapping threshold (T) and comfort (C) levels of the sound processing strategy when the implant is initially programmed. This correlation can be explained with reference to FIG. 22 where it can be seen that the NRT threshold levels are higher, although generally follow a similar pattern as the behavioural T-levels.

Preferably, the patterned threshold-reducing stimuli being applied, is below a psychophysically measured threshold. However, in other arrangements, the patterned threshold-reducing stimuli can be less than a behavioral measurement of perception threshold. This relationship is shown in FIG. 22, where it is apparent that psychophysical measures are less than behavioral measures.

For the purpose of advantageously adjusting the delivery of threshold-reducing patterned electrical stimulation, a feedforward processing block 121 can be provided as part of a feedforward path present in the speech processor unit 15. This feedforward path allows for the adjustment of the threshold reducing stimulation, based on the known behavior of the auditory system, through a suitable computational model, when referenced to the total or partial stimulation delivered during a known time period. An example of such a computational model is described below with reference to a “controlling algorithm” example.

Having determined the stimulation threshold, this information is then used to adjust the stimulation parameters to alter the stimulation as needed. Either only the characteristics of the threshold-reducing patterned electrical stimulation are modified, or alternatively, the characteristics of the whole stimulation pattern are altered to achieve the desired change in stimulation threshold. The preferred change in stimulation threshold is a reduction to the lowest threshold possible, the purpose being a reduction in the power consumed by the system. However, there are other types of changes that might be desirable, for example to localize the stimulation delivered to one particular set of neurons.

The threshold-reducing patterned electrical stimulation delivered by each electrode of the array may be varied depending on the measure of activity determined for that electrode over a preceding time period. This variation is made so that the overall stimulation received by the auditory fibers from any particular electrode over a predetermined period of time, is substantially equal to other auditory fibers receiving stimulation from other electrodes in the array.

A number of treatment regimes for the threshold-reducing patterned electrical stimulation are envisaged.

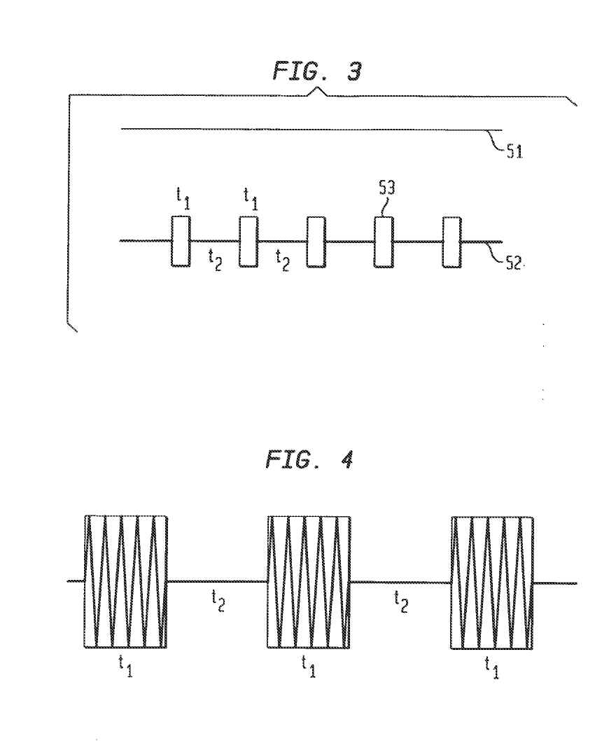

FIG. 3 is an exemplary depiction of patterned electrical stimuli as a function of time. Referring to FIG. 3, line 51 represents no auditory stimulation stimuli (line 51) being delivered. In parallel, regular occurrences of threshold-reducing stimuli 53 can be delivered to the cochlea 12.

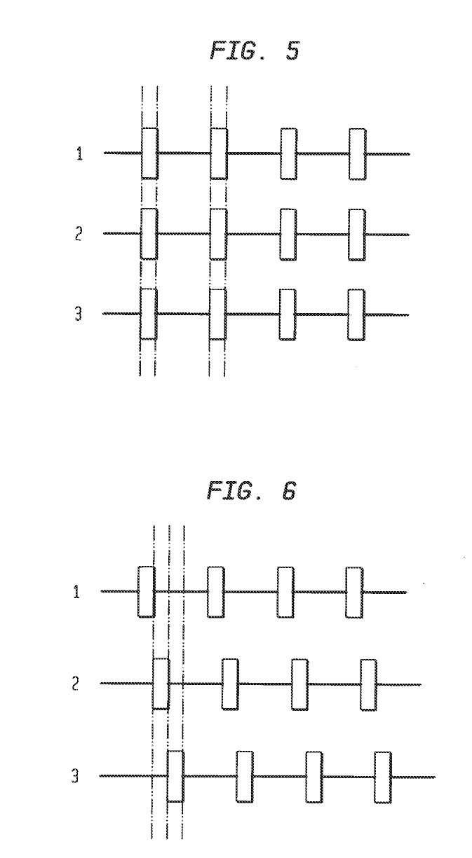

FIG. 4 is another exemplary depiction of patterned electrical stimuli as a function of time. Referring to FIG. 4, the threshold-reducing patterned electrical stimuli is delivered in a duty cycle comprising a period of time (t₁) of active stimulus and a period of time (t₂) of no stimulus. The total period of time between two stimulations (t₁+t₂) defines the duty cycle (DC), which is the basic unit of the stimuli. The duty cycles may be repeated, for each individual channel, in a sequence t₁−t₂, t₁−t₂, t₁−t₂, t₁−t₂ and so on.

A pause may be provided between duty cycles. The length of the pause may be variable. For example, a number of duty cycles may be applied in a sequence as mentioned earlier (t₁−t₂, t₁−t₂, t₁−t₂, t₁−t₂). Then, each group of such a plurality of cycles may be separated by a pause (a period of non-activity) t₃. For example, (t₁−t₂, t₁−t₂, t₁−t₂, t₁−t₂)−t₃−(t₁−t₂, t₁−t₂, t₁−t₂, t₁−t₂)−t₃−(t₁−t₂, t₁−t₂, t₁−t₂, t₁−t₂)−t₃−(t₁−t₂, t₁−t₂, t₁−t₂, t₁−t₂).

Each possible combination of the active stimulation time (t₁), no stimulation time (t₂) and pause between duty cycles (t₃), in addition to the auditory stimulation if present, is achieved through suitable modification of script of commands 117. There may be parameters in the command script 117 that are indirectly derived from the three times t₁, t₂ and t₃, with the calculation of these values performed at time of script creation.

Threshold reducing stimuli can provide a sharpening of special tuning curves, and/or provide a wider dynamic range in recipients of the IHPS. However, it should be appreciated that the efficacy of the treatment regime may depend on the one or more factors such as the length of deafness; the cause of deafness such as genetic, infection, ototoxic drug-induced, anatomy, for example, malformed cochlea; the morphology of the spiral ganglion cells; residual hearing; tonotopic organization; other treatments used before or after hearing loss, such as pharmacological, chemical, radiation, etc.; flow rate of the perilymph; diffusion properties of a delivered agent; existence of fibrous tissue around the scala tympani.

The delivery of the patterned electrical stimuli may be coincident with delivery of drug(s) for at least a period of time. Delivery of the stimuli and drug may be place and time specific, e.g., one type of drug and/or stimuli is applied to the basal part of the cochlea and another type of the drug and/or stimuli is applied to the apical part of the cochlea.

If more than one agent is administered, the administration of all drugs may be (1) uniform along the target organ, or (2) place and/or time specific, where at least one drug is preferably administered to one part of the target organ, e.g., the apical part of the cochlea, and another drug is preferably administered to another part of the target organ, e.g., the basal part of the cochlea. Administration of the drugs may occur simultaneously or at different times.

It should be appreciated that the agent delivered to the auditory system may be the desired agent which acts on the auditory system, or it may be a precursor for the desired agent that acts on the auditory system. The precursor for the desired agent may be in a form similar to that of the desired agent which undergoes a chemical, physical or biological change to take the form of the desired agent or may be an agent, action of which causes formation of the desired agent (e.g., gene injection where the gene itself is not the desired agent but activation of the gene produces the desired agent; e.g., a BDNF gene is not a desired agent but its action controls production and secretion of BDNF).

It should also be appreciated that more then one agent may be delivered to the auditory system. The agents may be delivered simultaneously, or sequentially, in predetermined manner.

The carrier member of the array may be coated with a slow-releasing film containing agents capable of reducing, directly or indirectly, firing thresholds of neurons. An initial dose of neurotrophic or other factors may be required to initiate the cell response which may be then maintained by patterned electrical stimulation. In addition or instead, the carrier member may be used to deliver neurotrophic factors to the site of implantation of the carrier member. In this regard, the implant may comprise a fluid reservoir and pump that is adapted to pump neurotrophic factors out of the carrier member and into the cochlea. An example of systems adapted to administer drugs are described in WO 03/072193 and WO 04/050056, each assigned to the assignee of the present application, and which is incorporated by reference herein.

An algorithm can be additionally or alternatively used to control the delivery of the patterned electrical stimulation, having more than one input. For example, one input can be a programming system to set desired parameters of the apparatus. Another input may rely on the results of special functions (Wₐ, Wₚ, Wₜ, Pₐ, Pₚ, Pₜ), where the index a refers to auditory stimulus, p for plasticity stimulus and t for threshold stimulus.

The algorithm used to control the delivery of the patterned electrical stimulation can also depend on feedback received by the apparatus, for example, whether auditory informative stimuli have been delivered, and the time that has elapsed since the last delivery of auditory informative stimuli. The type of stimuli may also depend on the overall stimulation level provided over a predetermined period of time, such as over one day.

The stimulating electrode array preferably includes a plurality of electrodes, each having a slightly different position with regard to the tissue of cochlea that is being stimulated. The patterned electrical stimulation may be applied to a single stimulating channel, some stimulating channels or all stimulating channels of the array. Further, when applied to multiple stimulating electrodes, the patterned electrical stimulation may be applied either simultaneously or sequentially with regard to the active part of the duty cycle.

In a simultaneous mode, multiple, if not all of the electrodes may be activated simultaneously, with the active part of the duty cycle being applied to all or some active channels, i.e., the active part of the duty cycle for each active electrode occurs simultaneously (as depicted in FIG. 5).

In another arrangement, if the stimuli are applied to multiple, if not all, stimulating electrodes, in a sequential mode, the active part of the duty cycle for one stimulating electrode occurs when all other electrodes are in the inactive part of the duty cycle, so at any given time only one stimulating electrode is active, as depicted in FIG. 6.

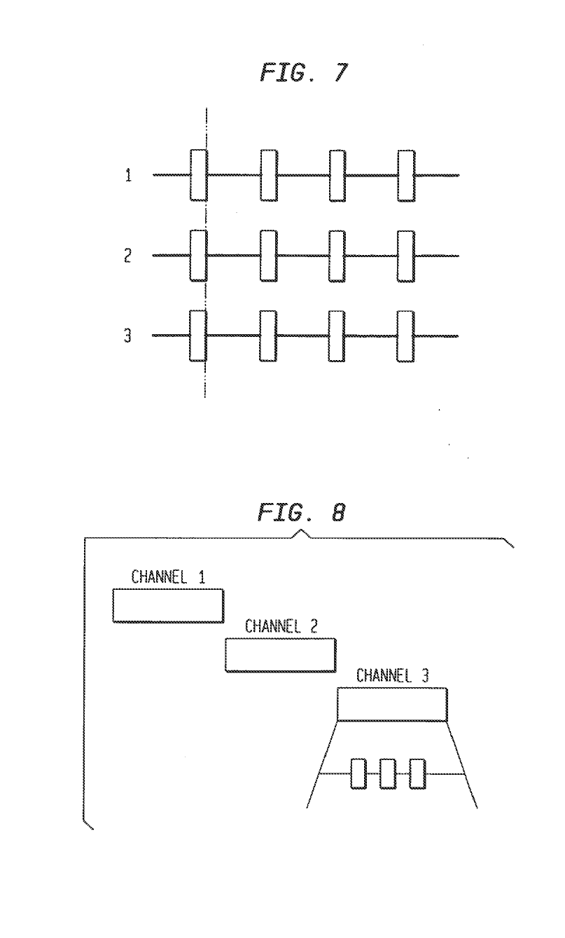

Still further, the stimuli may be applied to multiple if not all, stimulating electrodes, in a semi-sequential mode, where the beginning of the active part of the duty cycle for some or all stimulating electrodes is shifted in time so that the stimulation from one electrode occurs with a delay with respect to other stimulating electrodes, but before the active part of the duty cycle is finished, as is depicted in FIG. 7.

Still further, the threshold-reducing patterned electrical stimulation may be a combination of the above modes.

The IHPS 1 may comprise a first electrode array for delivering stimuli for reducing the firing threshold of neurons and a second electrode array for delivering auditory informative stimuli. In this regard, the first electrode array may be insertable into the neural network at a location different from that of the second electrode array.

In another example, threshold-reducing stimuli may be applied sequentially in which multiple duty cycles are delivered through one stimulating electrode before it is applied on another stimulating electrode of the array, as is depicted in FIG. 8.

The stimuli may be delivered when the implant is typically not in use, during a regularly occurring activity such as sleep, and/or sport activities, such as swimming. The apparatus measures the activity of one or more of the stimulating electrodes delivering auditory informative stimuli over a period of use, such as a day. For example, the apparatus may measure the frequency of stimulation or the stimulation current, used as input into the feedforward type system, previous described, and/or the neural response for each stimulating electrode, used as input into the feedback type system, also previously described. In this case, the apparatus may measure the different level of activity during the day exhibited by each of the electrodes and so provide a measure of the activity and/or the differences therebetween of the auditory fibers located along the cochlea.

Successful use of an inner ear prosthetic hearing implant is associated with a habituation process during which an inner ear prosthetic hearing implant recipient learns to interpret electrical signals presented by the implant as meaningful sound. Alternatively or additionally, the patterned electrical stimulation can be adapted to improve or maintain the plasticity of the neural system of the recipient as disclosed in the U.S. patent application Ser. No. 10/494,995, hereby incorporated by reference herein

In one embodiment, the algorithm used to control the delivery of the patterned electrical stimulation may be functional in two modes, i.e., acute and chronic. In the acute mode, the threshold-reducing stimuli may be delivered to the auditory system over a short period of time when compared to the length of time that the inner ear prosthetic hearing implant is active. In the chronic mode, the threshold-reducing stimuli may be presented over the same or comparable period of time as the length of time that the inner ear prosthetic hearing implant is active.

Each of many electrodes located at the intracochlear electrode array is tuned to the individual CI recipient and has its own behavioral T and C level. A decrease in firing thresholds, caused, for example, by threshold-reducing stimuli, will result in decrease in T levels for the recipient. Further, phycho-physical T levels can also be obtained through NRT measurements, as described previously. Therefore, the effects of applying subthreshold stimulation for the purpose of decreasing firing threshold of neurons can be shown by changes in T levels.

Simply, one could measure T levels before treatment of subthreshold stimulation—threshold reducing stimuli—and compare to T levels after the treatment. However, within this context, it should be noted that that T (and C) levels may change over time in either direction. There is a certain range of values within which T levels oscillate without apparent treatment being applied.

NRT can further be used to determine the selectivity for electric stimulation by measuring the spatial spread of electrically evoked neural excitation in the cochlea. This method involves a masker and a probe pulse on two electrodes. The probe position (electrode) is fixed, the masker position varies across the electrode array. The response amplitude is dependent on the overlap between the excitation regions of masker and probe. It is expected that the overlap depends on the stimulation current level, the mode of stimulation, placement of the electrode array relative to the neural fibers and the amount of surviving spiral ganglion cells.

A third method is to use psychophysical forward masking, which follows a similar masker—probe—principle as NRT. The masker is fixed in position and the current level and the probe is moved along the array. Another major difference is that it is not an objective measure but relies on the perceptive feedback of the CI recipient.

Alternatively, a controlling algorithm may be used. Here, for any stimulating electrode, Nᵢ, delivering auditory informative stimuli, a corresponding weighting function Wₐᵢ may be calculated according to:

Wₐᵢ=Σ(Tₐᵢ*Eₐᵢ*Nₐᵢ), i being between 1 and n (1)

where:
n is a total number of stimulating electrodes;
Nᵢ is the stimulating electrode for which the weight is being calculated;
Tᵢ is time of stimulus;
Eᵢ is amplitude of stimulus; and
Nᵢ is a contribution factor for the particular electrode; N₁ has the strongest contribution and electrodes positioned farther from N₁ have decreasing contribution but not necessarily in a uniformly decreasing manner.

A weighting function Wₜᵢ for the threshold reducing stimuli may be calculated:

Wₜᵢ=Σ(Tₜᵢ*Eₜᵢ*Nₜᵢ), i being between 1 and n (2)

where:
n is a total number of stimulating electrodes;
Nᵢ is the stimulating electrode for which weight is being calculated;
Tₜᵢ is time of stimulus;
Eₜᵢ is amplitude of stimulus; and
Nₜᵢ is a contribution factor for the particular electrode; N₁ has the strongest contribution and electrodes positioned farther from N₁ have decreasing contribution but not necessarily in a uniformly decreasing manner.

In a similar manner, a weighting function Wp for the plasticity-informative stimuli may be calculated:

Wpᵢ=Σ(Tₚᵢ*Eₚᵢ*Nₚᵢ), i being between 1 and n (3)

where:
n is a total number of stimulating electrodes;
Nₚᵢ is the stimulating electrode for which weight is being calculated;
Tₚᵢ is time of stimulus;
Eₚᵢ is amplitude of stimulus; and
Nₚᵢ is a contribution factor for the particular electrode; N₁ has the strongest contribution and electrodes positioned farther from N₁ have decreasing contribution but not necessarily in a uniformly decreasing manner.

In this way, the effect of direct stimulation is taken into account as well as the stimulation delivered by adjoining stimulating electrodes.

The auditory probability (Pₐᵢ) for each particular stimulating electrode to deliver threshold reducing stimuli can be expressed as a function of the weight (Wₐᵢ) of auditory informative stimuli:

Pₐᵢ=ƒ(Wₐᵢ)

This function that relates the weight of auditory informative stimuli and probability of delivering a threshold-reducing stimulus is complex.

The plasticity informative probability (Pₚᵢ) for each particular stimulating electrode to deliver plasticity informative stimuli is then a function of the weight (Wₚᵢ) of plasticity informative stimuli:

Pₚᵢ=ƒ(Wₚᵢ)

Further, the threshold-reducing probability (Pₜᵢ) for each particular stimulating electrode to deliver threshold-reducing stimuli is then a function of the weight (Pₜᵢ) of threshold-reducing stimuli,

Pₜᵢ=ƒ(Pₜᵢ)

The total probability for each particular stimulating electrode to deliver threshold-reducing stimuli is then a function of the weight of the auditory, plasticity inforative and threshold-reducing stimuli:

P=ƒ(Pₐ,Pₚ,Pₜ).

In another arrangement, auditory, plasticity informative and threshold-reducing stimuli may be delivered together or in combination. Alternatively, the auditory informative stimuli are superimposed on the threshold-reducing stimuli.

In another example, the system monitors the activity of the electrodes and determines the weight of the auditory informative stimuli, similar to the above formula. The probability of the stimulating electrode delivering threshold-reducing stimuli may be inversely proportional to the auditory informative stimuli weight and plasticity informative stimuli weight. The result is that the longer the period of time a neuron spends without being active (firing), the higher the probability that that stimulating electrode will deliver threshold-reducing stimuli to the auditory system, as shown by:

Pₓᵢ=ƒ(1/Wᵢ),Wₓᵢ=ƒ(tₓᵢ)

where:
Pₓᵢ is the probability of delivering threshold-reducing stimuli, related to a period of auditory informative stimulus inactivity,
Wₓᵢ is the weight of auditory informative stimuli and is proportional to the period of time without auditory informative stimuli tₓᵢ.

In one example, neural response telemetry (NRT) may be used to create a function, ƒ, which measures the neural activity as a response to a stimulating signal, and thus be provided as an input to the apparatus.

Overall, the probability of threshold-reducing stimuli, in a situation in which it is not predetermined, may be represented as a complex function that correlates to the activity of the implant and tissue. The electrical stimulation presented to the tissue may be: (i) Auditory Informative Stimuli, conveying auditory information; (ii) Plasticity Informative Stimuli, conveying plasticity information; or (iii) Threshold Informative Stimuli, conveying threshold reducing information.

Each of the activities may be measured as: (i) Electrical stimulation presented by the implant: (ii) Tissue response as measured by NRT; or (iii) Tissue response as measured by eABR.

Probability is a complex function:

P=ƒΣcᵢ*ΣPᵢ

where c, P have indexes a, p, t, as follows:

[P=ƒ{(c_{a(PIVTF)}×P_{a(PIVTF)}),(c_{a(NRT PIVTF)}×P_{a(NRT PIVTF)}),(c_{a(eABR PIVTF)}×P_{a(eABR PIVTF)}), (c_{p(PIVTF)}×P_{p(PIVTF)}),(c_{p(NRT PIVTF)}×P_{p(NRT PIVTF)}),(c_{p(eABR PIVTF)}×P_{p(eABR PIVTF)}), (c_{t(PIVTF)}×P_{t(PIVTF)}),(c_{t(NRT PIVTF)}×P_{t(NRT PIVTF)}),(c_{t(eABR PIVTF)}×P_{t(eABR PIVTF)})(c_{a(TIVTF)}×P_{a(TIVTF)}),(c_{a(NRT TIVTF)}×P_{a(NRT TIVTF)}),(c_{a(eABR TIVTF)}×P_{a(eABR TIVTF)}),(c_{p(TIVTF)}×P_{p(TIVTF)}),(c_{p(NRT TIVTF)}×P_{p(NRT TIVTF)}),(c_{p(eABR TIVTF)}×P_{p(eABR TIVTF)}),(c_{t(TIVTF)}×P_{t(TIVTF)}),(c_{t(NRT TIVTF)}×P_{t(NRT TIVTF)}), c_{t(eABR TIVTF)}×P_{t(eABR TIVTF)})}],

where:
c is a contributing coefficient for each of the probabilities; and
index a is related to auditory informative stimulus;
index p is related to plasticity informative stimulus;
index t is related to threshold reducing stimulus;
   The following indexes are applied, according to the input received from a particular variable tracking function (VTF).

PIVTF is related to a plasticity informative VTF;

NRT PIVTF is related to an NRT-based plasticity informative VTF;

eABR PIVTF is related to eABR-based plasticity informative VTF;

TIVTF is related to threshold informative VTF;

NRT TIVTF is related to NRT-based threshold informative VTF;

eABR TIVTF is related to eABR-based threshold informative VTF.

| | Auditory | Plasticity | Threshold |
|---|---|---|---|
| | Informative | Informative | Informative |
| | Stimulus | Stimulus | Stimulus |
| Function | Normal | Plasticity | Threshold |
| | function i.e., | Informative | Informative |
| | converting | Variable | Variable |
| | sound to | Tracking | Tracking |
| | electrical | Function | Function |
| | stimulation | (PIVTF) | (TIVTF) |
| | signals | | |
| Activity measured as | c_{a(PIVTF)} | c_{p(PIVTF)} | c_{t(PIVTF)} |
| electrical stimulation | c_{a(TIVTF)} | c_{p(TIVTF)} | c_{t(TIVTF)} |
| presented to the | P_{a(PIVTF)} | P_{p(PIVTF)} | P_{t(PIVTF)} |
| tissue (no index in | P_{a(TIVTF)} | P_{p(TIVTF)} | P_{t(TIVTF)} |
| the formula) | | | |
| Activity measured as | c_{a(NRT PIVTF)} | c_{p(NRT PIVTF)} | c_{t(NRT PIVTF)} |
| tissue response, | c_{a(NRT TIVTF)} | c_{p(NRT TIVTF)} | c_{t(NRT TIVTF)} |
| measured by NRT | P_{a(NRT PIVTF)} | P_{p(NRT PIVTF)} | P_{t(NRT PIVTF)} |
| (index NRT) | P_{a(NRT TIVTF)} | P_{p(NRT TIVTF)} | P_{t(NRT TIVTF)} |
| Activity measured as | c_{a(eABR PIVTF)} | c_{p(eABR PIVTF)} | c_{t(eABR PIVTF)} |
| tissue response, | c_{a(eABR TIVTF)} | c_{p(eABR TIVTF)} | c_{t(eABR TIVTF)} |
| measured by eABR | P_{a(eABR PIVTF)} | P_{p(eABR PIVTF)} | P_{t(eABR PIVTF)} |
| (index eABR) | P_{a(eABR TIVTF)} | P_{p(eABR TIVTF)} | P_{t(eABR TIVTF)} |

The present applicant hypothesizes that the relationship between the patterned electrical stimulation at subthreshold amplitudes and the reduction of thresholds is as follows: Subthreshold electrical stimulation, causes changes in biochemical cascades or processes. This results in changes in ion concentrations on two sides of the neuron membrane. This, in turn, causes a change in firing threshold of the neuron.

More particularly, it is suggested that the subthreshold patterned electrical stimulation influences influx of Ca²⁺ ions into cells. In turn, the membrane potential decreases due to change in ion concentration across the membrane. This phenomenon is addressed in K. Kimura et al., Journal of Biotechnology, Vol 63, 1998, pp 55-65: Gene expression in the electrically stimulated differentiation of PC12 cells, which is hereby incorporated by reference herein.

The neurotrophic factors that are released from the neurons by delivery of the threshold-reducing stimuli can be neurotrophic factors that also increase the survival of spiral ganglion cells. Such cells need to function if an implantee is to successfully use an IHPS.

The electrical stimulation may affect intracellular biochemical processes in a number of ways; for example, by releasing intracellular calcium ions (Ca²⁺) from intracellular storages, change in conductivity of the ion selective channels that control ion transport across the cell membrane, acting of neurotrophins as neurotransmitters, changes in cell (neuron) membrane that influence activity of ion channels, neurotrophic receptors, etc.

It should be apparent to those of ordinary skill in the art based on the description provided herein that an IHPS configured in accordance with the teachings of the present invention is capable of delivering patterned electrical stimulation, specifically to elicit endogenous secretion of neurotropic factors and/or other factors from neurons, in such a way as to reduce firing thresholds of neurons. These naturally occurring substances have a capacity to activate the neurotrophic receptors. For example, adenosine is known to activate neurotrophic receptors.

The naturally occurring agent that is produced and/or released may be one or more neurotrophic factors (or neurotrophins), such as Brain Derived Neurotrophic Factor (BDNF), NGF (nerve growth factor), NT-3 (neurotrophin-3), NT-4/5 (neurotrophin-4/5), NT-6 (neurotrophin-6), LIF (leukemia inhibitory factor), GDNF (glial cell line-derived neurotrophic factor), FGF (fibroblast growth factor), CNTF (ciliary neurotrophic factor), and IGF-I (insulin-like growth factor-I).

Neurotrophic factors produce their effects on neurons by binding to neurotrophic receptors, such as trk receptors and a glucoprotein termed p75. The receptors span the plasma membrane. The extracellular part of the receptor molecule contains binding sites for neurotrophins. The intracellular part of the receptor features an enzyme active structural element, i.e., a tyrosine kinase. There are three known trk proteins, termed trkA, trkB and trkC that preferentially bind NGF, BDNF and NT-4/5, and NT-3, respectively. It is generally assumed that neurotrophins are synthesized and packaged into vesicles in the soma in direct proportion to its mRNA, and that they are then transported to either presynaptic axon terminals or postsynaptic dendrites for local secretion. The secreted neurotrophins bind to and activate trk receptors in the pre- and post-synaptic membranes. Neurotrophin NT-3 also binds to trkB but with much less specificity than to trkC. Binding of the neurotrophins to the trk receptors leads to receptor tyrosine phosphorylation. The phosphorylation process triggers the activation of molecular cascades or pathways that control cell functioning. At the same time, binding of the neurotrophins to receptor p75 is non-specific. By itself, the receptor is unable to mediate any neurotrophin actions, but its presence is required for certain cell functions, most notably apoptosis.

Neurotrophic factors are a key element in a number of essential cell processes such as cell growth, cell apoptosis (programmed death), and functionality of various cell organeleas. In addition to this, neurotrophic factors have more specific functions in neurons: controlling functionality of ion channels that determine membrane potential which, in turn, controls the neural firing properties of the cell, establishment and maintenance of synapses, etc.

Neurotrophins secreted by the postsynaptic cell are likely to be highly localized owing to their propensity to bind to the cell surface near the secretion site. Endogenous neurotrophins, secreted in response to synaptic activity, induce the morphological changes that lead to the maintenance of the existing synapses or formation of new synaptic contacts.

In the absence of signals, synaptic contacts may disconnect, breaking the particular neural pathway. Synaptic action of neurotrophins consists of two modes. In a resting “permissive” mode, neurotrophins are secreted at a low level through constitutive secretion or regulated secretion triggered by subthreshold and low-frequency synaptic activity. This permissive mode provides trophic regulation of synaptic functions, including the ability to generate long-term potentiation. In the active “instructive” mode, neurotrophic factors are secreted as a higher level of response to intense synaptic activity that results in a transient high-level calcium concentration in the post-synaptic cytoplasm.

In an alternative arrangement, the stimulator device may be housed in a housing that is totally implantable within the implantee. In this case, the housing further houses a power source that provides the apparatus with at least sufficient power to deliver stimuli for reducing the firing threshold of neurons.

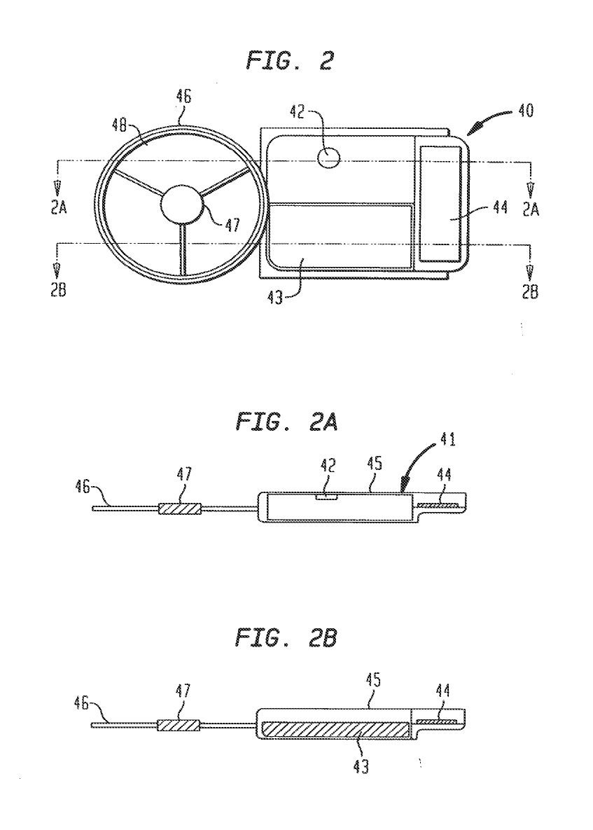

FIGS. 2, 2a and 2b are different views of a totally implantable IHPS receiver/stimulator package which is capable of operation, at least for a period of time, without reliance on components worn or carried external to the body of the implantee. An example of the structure and function of a totally implantable prosthetic hearing system is described in International Application No. PCT/AU01/00769, the entire contents and disclosure of which is hereby incorporated by reference.

Implant 40 is adapted for implantation in a recess formed in the temporal bone adjacent the ear of the implantee that is receiving the implant. Implant 40 may be implanted in a manner similar to how the receiver/stimulator unit 22 shown in FIG. 1 may be implanted.

In another example, the stimuli is delivered to the Cochlea Nucleus (CN), for example, via an auditory brainstem implant (ABI) or PABI electrode.

FIG. 10 is a simplified drawing of an alternative apparatus 100 that is adapted to deliver threshold-reducing stimuli to the CN. The apparatus 100 has a housing 101 for a stimulator device and an electrode array 102 extending therefrom. As shown, the electrode array 102 may comprise a plurality of electrodes 103. In this illustrative embodiment, the stimuli may be delivered to the inferior colliculus. For example, apparatus 100 may be provided with a Mid-Brain Implant (MBI) wherein the stimulating electrode is positioned adjacent the inferior colliculus to apply the appropriate stimulus.

In an alternative arrangement, the stimuli is delivered to the cochlea via an endosteal electrode array. Generally, an endosteal electrode array is not inserted into the scala tympani, but rather into a natural crevice in the cochlea that allows for the hydrodynamic nature of the cochlea to be maintained. An example of an endosteal electrode array is described in WO 02/080817, which is hereby incorporated by reference herein.

In an alternative embodiment, the apparatus may be adapted to deliver stimuli to the auditory system of the implantee, where the hearing prosthesis is a middle ear implant.

While the above description has concentrated on describing use of a modified inner ear prosthetic hearing implant to deliver the threshold-reducing stimuli, it should be understood that such stimuli may be delivered using a device that is implanted in conjunction with or instead of an inner ear prosthetic hearing implant. Further, the apparatus may be installed to deliver patterned electrical stimulation to the cochlea of a recipient that is not receiving the inner ear prosthetic hearing implant. For example, delivery of threshold-reducing stimuli may be performed in conjunction with use of a middle ear implant or a hearing aid.

The delivery of the patterned electrical stimuli may occur at times when the apparatus is incapable of, or is not delivering auditory informative stimuli. For example, the delivery of threshold-reducing stimuli may occur when the implantee is asleep and not using the apparatus for the delivery of auditory informative stimuli. Referring to FIG. 3, no auditory stimulation stimuli (line 51) is being delivered to cochlea 12 and at this time, regular occurrences of threshold-reducing stimuli 53 are being delivered to cochlea 12.

The electronics housed in the implantable unit is provided with a clock, controlling the overall operation of the device. This clock may control the timing with which the predetermined stimulation pattern may occur. This clock may be programmable to operate in “real time” such that the recipient or implantee may receive threshold-reducing stimuli at times when the recipient is asleep or not receiving auditory informative stimuli.

To treat problems with the visual system, a stimulus may be delivered to the retina or visual cortex in patients suffering from loss of vision. In this regard, retinal and visual cortex implants are the two most commonly investigated devices for applying such stimulation for the visually impaired. In this configuration, the apparatus may be adapted to solely deliver patterned electrical stimuli for reducing the firing threshold of neurons to the visual system, or for providing plasticity informative stimuli. Similarly, when the apparatus is delivering stimuli to the visual system, the patterned electrical stimulation may have a magnitude less than the visual perception threshold of the implantee.

Further, stimulation may be delivered to the Subthalamic Nucleus (STN), the Globus Pallidus (GPi), and/or the Thalamus of the implantee. Such stimulation may be administered via deep brain stimulation.

The relationship between patterned electrical stimulation and the release of Endogenous Brain-Derived Neurotropic Factor (EBDNF) by the central neurons is discussed in Cellular Mechanisms Regulating Activity-Dependent Release of Native Brain-Derived Neurotropic Factor from Hippocampal Neurons, Journal of Neuroscience, Vol 22, 2002, pp 10399-407, Balkoweic A and Katz D. M., which is hereby incorporated by reference herein.

### Example 1

An animal model was established to demonstrate the ability of acute administration of BDNF to modulate thresholds in deafened guinea pigs.

A guinea pig is a widely used animal model for studying function as well as dysfunction of the auditory system. The guinea pigs used in the examples were deafened by administration of ototoxic drugs. These drugs have the ability to destroy hair cells, leading to sensorineural hearing loss.

A typical experimental set-up involves implantation of an animal inner ear stimulator and measurements of auditory brainstem response (ABR) as a function of electrical stimuli delivered by an intracochlear electrode array. The electrode array was implanted into the cochlea and connected to an external stimulator which supplied electrical stimulation. eABR recordings were made by a separate recording system, using electrodes positioned at the skull and neck of the guinea pig. Recording was conducted through a separate pair of electrodes, positioned away from the cochlea and close to brain: one on the skull and other in the neck. Frequently, ABR elicited by electrical stimulation is also referred to as electrically evoked auditory brainstem response (eABR).

A range of electrical stimuli delivered by the intracochlear electrode array was typically between 50 and 2000 μA delivered as 100 μs biphasic pulses. The auditory brainstem response is measured in μV, where a typical eABR response has a wide range from sub-micro V to tens of μV.

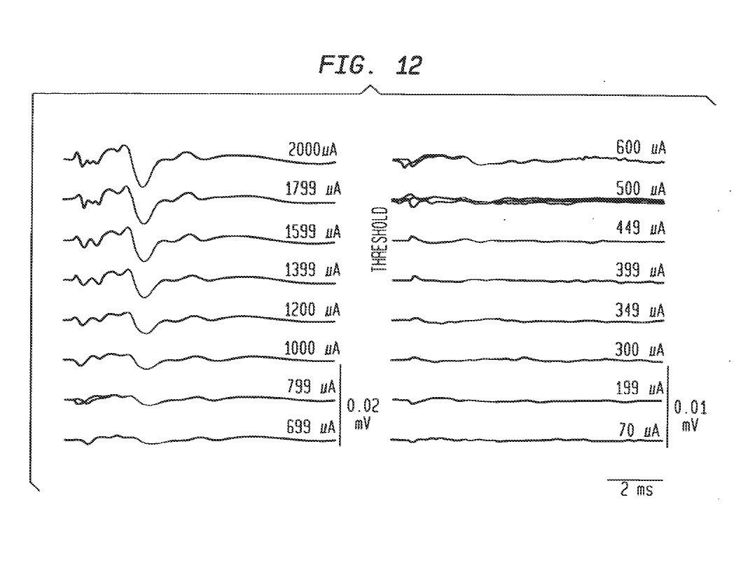

A typical eABR has a very complex shape, featuring several peaks, corresponding to activity of various parts of the brainstem, as shown in FIG. 12.

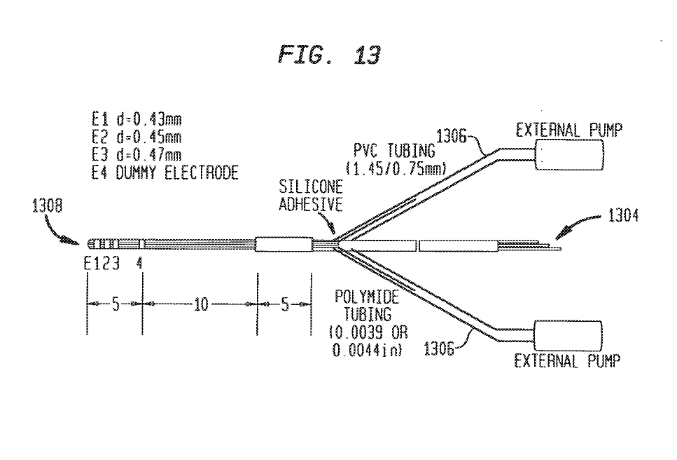

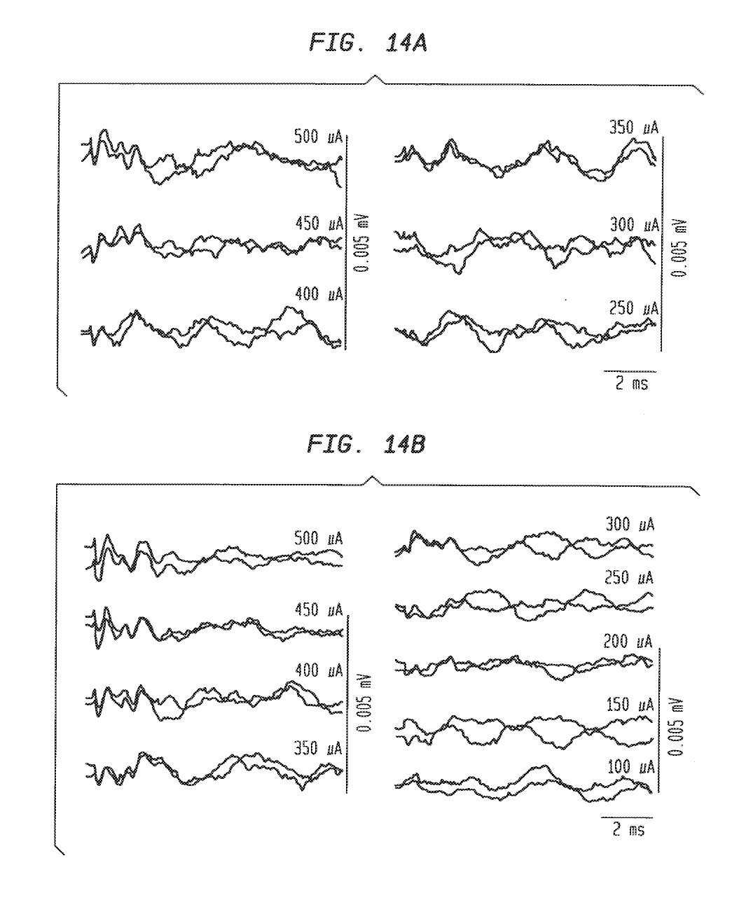

Referring to FIG. 13, the present example used a custom made guinea pig intracochlear electrode array featuring three stimulating electrodes 1302, connected to lead wires 1304, and one or two delivery tubes 1306, positioned inside the silicone body of the array. The tubes protrude to the very tip of the array so delivery of the agents occurs at the very end of the apical end 1308 of the array. On the opposite end, the tubes are connected to independent syringes containing desired solutions. The delivery rate for each syringe is controlled by a micropump, which very precisely delivers quantities from nL/min to μL/sec.

The present examples used solutions of: artificial perilymph (RAP), and a naturally occurring molecule, Brain Derived Neurotrophic Factor (BDNF). Artificial perilymph is used to mimic naturally occurring perilymph because the two have a similar chemical content. Thus, the artificial perilymph is used as a control and should not change thresholds.

The content of the experimental artificial perilymph (RAP), as well as a solution of BDNF, being created from a RAP solution in which BDNF was dissolved, is shown in Table 1:

TABLE 1
Content of various solutions used to perfuse the
cochlea throughout experiments.

| Concentration in (mM) | RAP | BDNF |
|---|---|---|
| Sodium | 148 | 148 |
| Potassium | 4.2 | 4.2 |
| Chloride | 133.8 | 133.8 |
| Bicarbonate | 21 | 21 |
| Calcium | 1.3 | 1.3 |
| BDNF (ug/mL) | 0 | 100 |

For the experimental work described in the present examples, the left ear of a guinea pig was used as the location to implant the electrode array with two drug delivery channels.

In some experiments, the right ear was implanted with an “ordinary” animal electrode array featuring three electrodes and no drug delivery channels. The surgery and implantation were performed exactly as for the left ear. The intention was to use the right ear as a control against which the effects of various biochemical agents perfused in the left ear on the response of that auditory system could be measured.

It was assumed that the response from the right ear, which was not challenged, would be relatively constant, within boundaries of natural noise. Recordings from the right ear were taken more sporadically than from the left ear. Indeed, according to expectations, the eABR response from the right ear in a course of the experiment was very stable and the variations observed were not related to the perfusion of various agents in the left ear.

The identified procedure was applied to 8 animals: 3 perfused with artificial perilymph (RAP), and 5 perfused with BDNF.

| Subject | Agent | Abbreviated |
|---|---|---|
| 3 x guinea pig, 4 weeks deaf | Artificial perilymph | RAP |
| 5 x guinea pig, 4 weeks deaf | BNDF (100 ug/mL) in | BDNF |
| | artificial perilymph | |

The results of the experiments including the recorded responses for thresholds, of eABR response are shown in Table 2 below.

TABLE 2
Quantitative changes in thresholds as a result of the administration
of various pharmacological agents: RAP and BDNF, into the left
cochlea over a period of one hour.

| | | | Threshold | | |
|---|---|---|---|---|---|
| | DoD | | (uA) | | |
| Agent | (wk) | Ear | Before | After | Comment |
| RAP | 14 | L | 350 | 350 | No change (<1 h) |
| | | R | N/A | N/A | |
| RAP | 4 | L | 175 | 175 | No change (<1 h) |
| | | R | N/A | N/A | |
| RAP | 4 | L | 600 | 600 | No change (<1 h) |
| | | R | 500 | 500 | |
| BDNF | 4 | L | 400 | 250 | Sharp decrease (<10 min) |
| | | R | 600 | 600 | |
| BDNF | 4 | L | 250 | 200 | Slow and small change, (>1 h) |
| | | R | 300 | 300 | |
| BDNF | 4 | L | 350 | 150 | Sharp decrease (<10 min) |
| | | R | 550 | 550 | |
| BDNF | 4 | L | 300 | 200 | Sharp decrease (<10 min) |
| | | R | 300 | 300 | |
| BDNF | 4 | L | 550 | 300 | Sharp decrease (<10 min) |
| | | R | N/A | N/A | Right, control ear was |
| | | | | | not implanted |

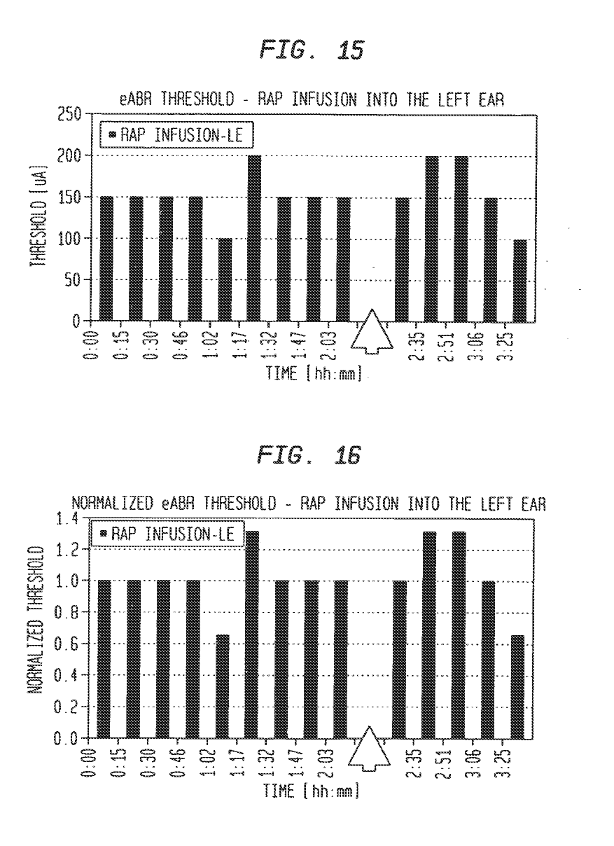

First, a stable eABR threshold was recorded for the implanted, typically left, ear without any perfusion of any agents. Stable recordings were obtained over at least an hour, as shown in FIGS. 15 and 16. FIG. 15 shows absolute values and FIG. 16 shows normalized values.

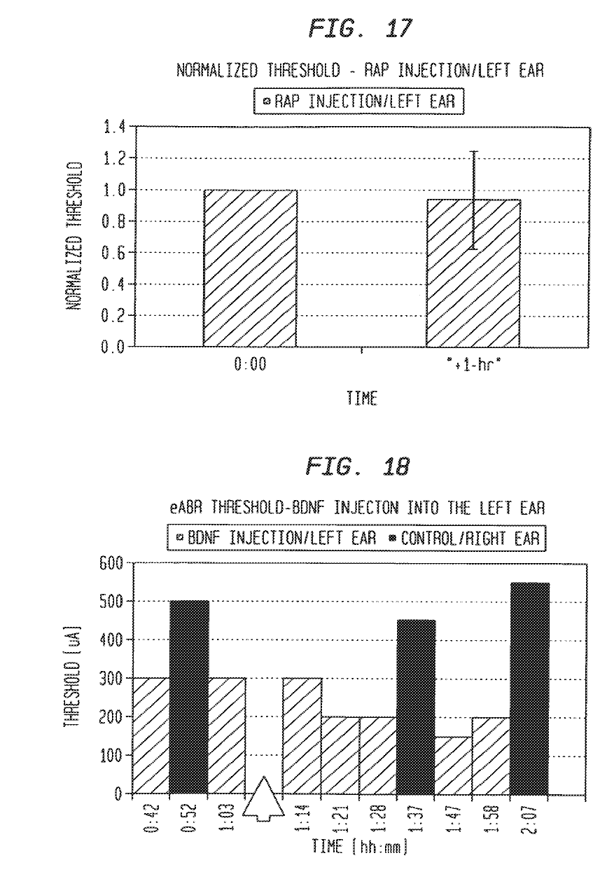

Then, the guinea pigs were infused with RAP, and eABR thresholds were measured over a period of 1 hour. No change in the eABR was observed, indicating that the RAP did not influence neural response. This is summarized in FIG. 17, where normalized values for eABR before and after perfusion of RAP are compared.

Animals perfused by BDNF showed a clear decrease in threshold for the left ear, immediately following perfusion of BDNF, with the minimum achieved within 30 minutes of perfusion. Further perfusion did not lower the threshold. The value of the threshold was roughly halved. At the same time, the threshold for the right ear stayed stable, unaffected by perfusion of the agent solution in the left ear.

A typical response is shown in FIG. 18. FIG. 18 shows the eABR response prior to infusion of any BDNF in the system and the response after BDNF was perfused for 30 minutes. The change in threshold is evident. The threshold before perfusion was 300 μA. As soon as the BDNF solution was introduced, the threshold started dropping and reached its minimum, at 150 μA after 30 minutes when the recording in FIG. 18 was taken.

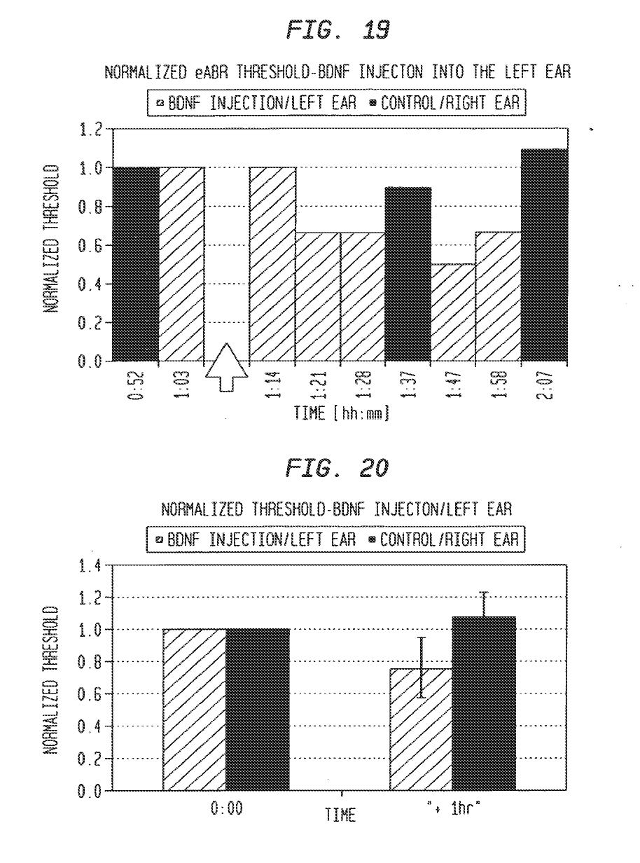

The change of the threshold over time is shown in FIGS. 18 and 19. Stable eABR threshold readings were obtained prior to perfusion of BDNF. Shortly after perfusion of BDNF, a substantial decrease in eABR threshold was observed. At the same time, thresholds recorded in the right ear maintained its value. FIG. 18 shows absolute values and FIG. 19 shows normalized values for the eABR thresholds.

A summary result is provided in FIG. 20 which shows normalized eABR thresholds remained stable in the right ear both before and during infusion of the chemical agents into the left ear. In the left ear, eABR thresholds sharply decreased shortly after addition of BDNF.

It is important to point out the features which make these experiment results outstanding. Reference is made, for these comparisons, to Takayuki Shinohara et al., Neurotrophic factor intervention restores auditory function in deafened animals, Proceedings of the Academy of Science of the USA, Feb. 5, 2002, Vol. 99, No. 3, pp. 1657-1660; and R. K. Shepherd et al., Protective Effects of Patterned Electrical Stimulation on the Deafened Auditory System, NIH, Eighth Quarterly Progress Report, NIH-N01-DC-0-2109, Jul. 1-Sep. 30, 2002. Both references are hereby incorporated by reference herein.

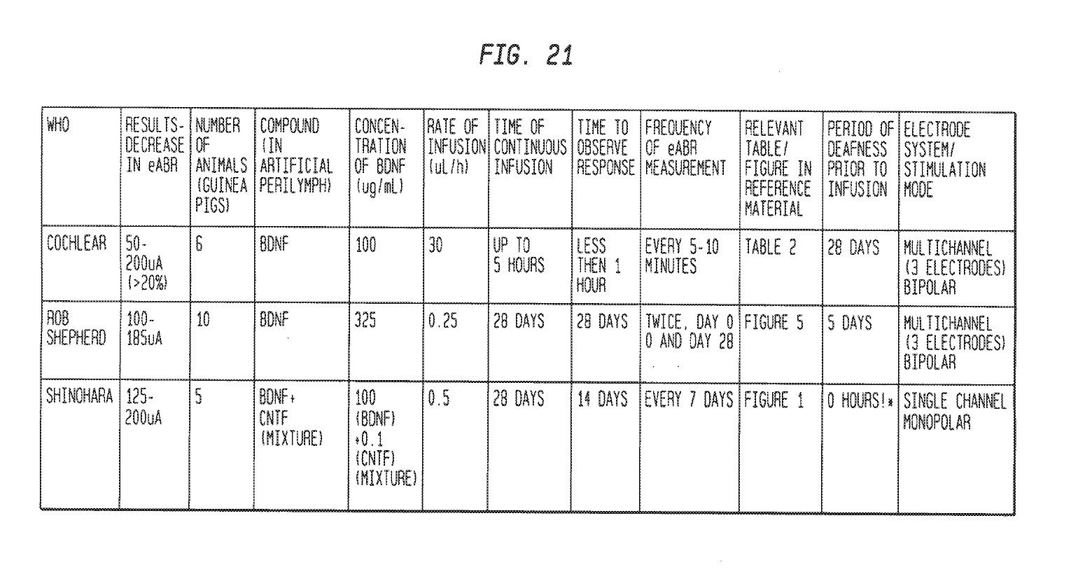

Infusion of the BDNF started, in one experiment, after 28 days of deafness. Other experiments have been conducted where the guinea pig was deafened for shorter periods before starting the experiments. For example, 5 days by Shepherd and 0 days by Shinohara (see FIG. 21). Hence, in the present applicant's experiments, there was a relatively long period where the loss of the spiral ganglion cells, following destruction of the hair cells, has become substantial.

Further, the present applicant observed a response after a short period of time, less that an hour, typically in order of minutes after infusion of BDNF. The work of Shepherd and co-workers measured eABR responses only twice, at the beginning (time 0) and end (day 28), thus suggesting that they were not expecting to see acute effects of BDNF on eABR thresholds.

Concentration of the agent (BDNF) was comparable in all three cases. What made the difference is significantly higher rate of infusion, two orders of magnitude higher than in other two labs. Over 1 hour, the present applicant infused 3 μg of BDNF, Shepherd and co-workers infused 0.08 μg (˜2.7% of the total infused by embodiments of the present invention) and Shinohara and co-workers 0.05 μg (1.7% of the total of embodiments of the present invention). These results are summarized in FIG. 21.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

For example, the application of the apparatus is not limited to the auditory system and may be successfully used to treat other conditions caused by the lack of natural functionality or abnormal function. For example, spinal cord injury, visual impairment, sensorineural and motorneural abnormalities, such as depression, Parkinson's disease, Alzheimer's disease may also be treated with the herein described device. For the treatment of spinal cord injured patients, the stimulus can be delivered to various locations along the patient's spinal cord. An example of a functional electrical stimulation device is described in WO 02/013694, which is hereby incorporated by reference herein.

All documents, patents, journal articles and other materials cited in the present application are hereby incorporated by reference.

1.-86. (canceled)

87. An apparatus for delivering electrical stimuli to an implantee, comprising:
a stimulator configured to generate electrical stimulation signals having the stimuli encoded therein; and
an electrode array configured to be implanted in the auditory system of the implantee, the electrode array having electrode members configured to deliver the electrical stimulation signals to the implantee;
wherein the stimuli includes neuron firing threshold-reducing stimuli configured to trigger the production and/or release of naturally occurring chemical agents to cause a reduction in the firing thresholds of neurons,
wherein the neuron firing threshold-reducing stimuli has a magnitude below both perception and psychophysical thresholds.

88.-98. (canceled)
